(19) United States
(12) Patent Application Publication
(10) Pub. No.: US 2009/0317468 A1
(43) Pub. Date: Dec. 24, 2009

(54) MULTICOMPARTMENT GRANULATE FORMULATIONS FOR ACTIVE SUBSTANCES

(75) Inventors: Claudia Letmathe, Bergisch Gladbach (DE); Axel Eble, Berlin (DE); Olaf Bork, Stuhr (DE)

Correspondence Address: NORRIS, MCLAUGHLIN & MARCUS, PA, 875 THIRD AVENUE, 18TH FLOOR, NEW YORK, NY, 10022, US

(73) Assignee: BAYER TECHNOLOGY SERVICES GMBH, Leverkusen (DE)

(21) Appl. No.: 12/374,568

(22) PCT Filed: Jul. 7, 2007
(86) PCT No.: PCT/EP2007/006046

§ 371 Date: Mar. 13, 2009

(30) Foreign Application Priority Data
Jul. 21, 2006 (DE) 10 2006 033 723.9

(51) Int. Cl. A61K 9/00

(52) U.S. Cl. 424/484

(57) ABSTRACT

The invention relates to molded article that contains active substances and comprises at least two compartments that have a different material composition. Each compartment is independently provided with at least one active substance that is contained in a matrix. Each matrix encompasses at least one filler at a concentration of ≧20 percent by weight to ≦100 percent by weight relative to the total weight of the respective matrix. The invention further relates to a method for producing such molded articles as well as the use thereof.

The present invention relates to a molded article comprising active substances which comprises at least two compartments with different material composition, each compartment exhibiting, independently of one another, at least one active substance. The present invention furthermore relates to a process for the preparation of such molded articles and to the use thereof.

Granules comprising active substances at present more often than not comprise one or more active substances formulated with inorganic fillers. Processes such as pan or fluidized bed agglomeration/granulation and also low pressure extrusion and spray drying are applied in the preparation of such granules.

However, it is desirable, with different fields of application, for example, in the use of agrochemicals, pharmaceutical active substances or veterinary medicine active substances, to formulate a combination of spatially separated active substances as granules, in particular as rapidly water-dispersible granules. However, it is frequently impossible to charge two different granules with one active substance each or one other substance each and to apply the granules as a mixture. This type of formulation can result in segregation in the product container and accordingly in unintentional active substance concentrations when applied.

DE 100 22 989 A1 describes the use of a combination of an agrochemical active substance and a solid support enclosing the active substance in the suppression of antagonistic interactions in a mixture of the active substance enclosed by the support and at least one additional agrochemical active substance. Preferred formulations comprising such a combination comprise herbicides combined with a support, together with a safener and/or a growth regulator. The formulations make it possible to suppress antagonistic interactions between different active substances.

However, this document is geared to the encapsulation of an active substance in microcapsules. It is a disadvantage here that such an encapsulation brings about additional costs and expenditure in the preparation. The rapid redispersion of the capsules is not mentioned.

WO 1999/056540 discloses a solid composition of a plant protection agent which can be obtained by melt extrusion and forming of a mixture of 0.1-80% by weight of an active substance used in plant protection or a combination of such active substances, 10-80% by weight of at least one mineral filler, 0-20% by weight of inorganic or organic additives and to 100% by weight of at least one water insoluble thermoplastic polymer. This document furthermore discloses that a coating can be formed by coextrusion or postcoating, for example in a fluidized bed, using solutions or dispersions if appropriate comprising active substance and polymer, which coating varies the release of the molded article or comprises an additional active substance component.

However, this document does not disclose the more detailed composition of the outer coating. Furthermore, it is geared to the sedimentation of the granules and especially not to rapid redispersibility.

Accordingly, there furthermore exists, in the state of the art, the need for formulations of spatially separated active substances in molded articles, which can be rapidly redispersed.

The present invention has set itself the target of rectifying at least one of the above-mentioned disadvantages of the state of the art. In particular, it has set itself the target of making available a formulation of active substances in molded articles with several compartments which can be easily produced, which can be obtained with economic starting materials and/or which can be quickly redispersed in water, for example, in less than 600 seconds.

The object according to the invention is achieved by a molded article comprising active substances which comprises at least two compartments with different material composition, each compartment exhibiting, independently of one another, at least one active substance and the active substance being present in the individual compartments in each case in a matrix and the respective matrix comprising at least one filler, the filler content of the respective matrix, based on the total weight of the respective matrix, coming to ≧20% by weight to ≦100% by weight.

The term “active substance”, within the meaning of the present invention, describes substances or mixtures of substances, which have an effect desired by the user on an organism, irrespective of whether this organism is of human, animal, plant or other nature.

The term “molded article” within the meaning of the present invention, describes first any solid body which is hard, waxy elastic, amorphous or crystalline at ambient temperature, but which is not or not yet present in the liquid physical state. The molded article can, for example, be prepared by extrusion, coextrusion or compression.

The term “compartment” within the meaning of the present invention, describes a defined region of the molded article which can be distinguished from other regions of the molded article.

The term “matrix” within the meaning of the present invention, describes the substance or substance mixtures enclosing the active substance. The matrix can comprise fillers, binders, dispersants, swelling or bursting agents, preservatives, lubricants, antioxidants, antifoaming agents, wetting agents, surfactants and/or softeners or plasticizers.

The term “filler” within the meaning of the present invention, describes in particular an inert organic or inorganic material. The filler can in particular be inert with regard to the active substance used. The filler can be the same for the individual components of the molded article or can be different with individual compartments.

The advantages of a molded article according to the present invention are that the end user obtains a product with a precisely tailored active substance combination which the can likewise precisely meter. The fillers used are inexpensive to obtain. The handling thereof is known and can be easily adjusted for the desired result each time. An additional advantage is that the problem of the segregation of formulations with different molded articles is avoided. Through the choice of the matrix components, it is possible to produce products which rapidly redisperse in water, for example in the preparation of a spray slurry, an ointment or a drink. It is possible, with molded articles according to the invention, to obtain redispersing times for example of ≦600 seconds. If a partial rapid redispersibility is desired, it is possible, through the choice of matrix components, to obtain products with a controlled-release characteristic which releases over a relatively long period of time, on an agricultural effective area or on a target organism, the active substance combination hitherto not accessible as a result of incompatibilities.

It is likewise possible for the filler content of the respective matrix, based on the total weight of the respective matrix, to come to ≧40% by weight to ≦90% by weight or ≧70% by weight to ≦80% by weight.

In the context of the present invention provision is furthermore made, for the active substances used in the different compartments of the molded article to be incompatible, and thus together antagonistic. This means that at least one of the active substances would experience disadvantageous changes in direct contact with other active substances. Direct contact can, for example, result from a mutual presence in the solid phase. The disadvantageous changes may start quickly or may arise only in the course of relatively lengthy storage.

Parameters, by which incompatibilities of active substances can be described, are, for example, the hydrophilicity/hydrophobicity (expressed through the 10 g K_{OW} values), acid or base strength (expressed through the pKₛ/pK_{b} values) and solubility products of salts of anions of an active substance and cations of the other active substance (in order to describe the precipitation of sparingly soluble salts).

It is likewise possible, geared to the individual case, for special active substance molecules to enter chemical reactions with one another.

An additional advantage of the present invention is that even active substances incompatible with one another per se can be brought together in a single molded article if the filler content of the respective matrix, based on the total weight of the respective matrix, comes to ≧20% by weight to ≦100% by weight. Without being committed to a specific theory, it is assumed that the active substances, as a result of the high filler content in the compartments, are present in such a diluted form that an at most insignificant change in the active substances takes place on the surfaces of the compartments, but the active substances survive unchanged in the body of the compartments. In this connection, the filler also serves to prevent the diffusion of the active substances.

Naturally, the molded articles according to the invention or the active substances present in the individual compartments are already protected against incompatibilities during the preparation of the article.

In an embodiment of the present invention the total weight of the matrices, based on the total weight of the molded article, comes to ≧0.01% by weight to ≦99.99% by weight, preferably ≧1% by weight to ≦50% by weight, more preferably ≧4% by weight to ≦10% by weight. Such matrix proportions make it possible, as a result of the density of the materials used, to employ, in the preparation of a spray slurry or drink, molded articles which rapidly sink and accordingly are quickly dispersed.

In an additional embodiment of the present invention, the molded article comprises at least one first and one second compartment, the ratio by weight of the two compartments to one another coming to a ratio of ≧1:1 to ≦99:1, preferably ≧2:1 to ≦80:1, more preferably ≧5:1 to ≦50:1. If the molded article according to the invention exhibits compartments with such ratios by weight, the active substances can be released from the two compartments in a desired time ratio. For example, the active substance of the first component can be released much more quickly than that of the second. An additional example is the desired virtually simultaneous release of the active substances.

In an additional embodiment of the present invention, the molded article comprises at least one first and one second compartment, the ratio by weight of the total amount of the active substances to the total amount of the fillers in the first compartment coming to ≧0.01:99.99 to ≦99.99:0.01, preferably ≧1:50 to ≦50:1, more preferably ≧1:5 to ≦5:1 and the ratio by weight of the total amount of the active substances to the total amount of the fillers in the second compartment coming to ≦0.01:99.99 to ≦99.99:0.01, preferably ≧1:50 to ≦50:1, more preferably ≧1:5 to ≦5:1. The choice of such concentrations of the active substances in the fillers advantageously results in the active substances being so widely diluted in the inert medium that they cannot react significantly in a harmful fashion with one another or with external reactants, such as atmospheric oxygen, light and the like.

In an additional embodiment of the present invention, the active substances are agrochemical active substances, preferably chosen from the group consisting of herbicides, insecticides, fungicides, nematicides, pesticides, molluscicides, acaricides, fertilizers, growth regulators, safeners, bioenhancers and/or bird repellants. The term “agrochemical active substances” describes substances or substance mixtures, which are used in noncommercial or commercial farming, in order to achieve a desired result with regard to plant growth, pest infestation or the like. The abovementioned substance categories cover the active substances most frequently used in farming and are accordingly the most asked after by the user. It is possible to have present, in the individual compartments of the molded article according to the invention, in each case active substances of the same substance category but also to use, in the individual compartments, active substances from different categories.

Examples of herbicides can be chosen from the group consisting of:

2,3,6-TBA, 2,4,5-T, 2,4,5-TB, 2,4-D, 2,4-D-butotyl, 2,4-D-diolamine(2,4-D-diethanolamine), 2,4-D-isoctyl, 2,4-D-trolamine(2,4-D-triethanolamine), 2,4-D-2-ethylhexyl, 2,4-D-butyl, 2,4-D-dimethylammonium, 2,4-D-isopropyl, 2,4-D-sodium, 2,4-DB, 2,4-DB-isoctyl, 2,4-DB-butyl, 2,4-DB-dimethylammonium, 2,4-DB-potassium, 2,4-DB-sodium, 2,4-DEP, 2-phenyl-4H-3,1-benzoxazin-4-one, 4,5,7-trichloro-2,1,3-benzothiadiazole, 5-methyl-6-thioxo-1,3,5-thiadiazinan-3-ylacetic acid,
ACD 10614, acetochlor, acifluorfen, acifluorfen-sodium, aclonifen, acrolein, AKH-7088, alachlor, allidochlor, alloxydim, alloxydim-sodium, ametridione, ametryn, amibuzin, amicarbazone, amidosulfuron, amiprofosmethyl, amitrole, ammonium sulfamate, anilofos, anisuron, asulam, asulamsodium, atraton, atrazine, azafenidin, azimsulfuron, aziprotryn,
barban, beflubutamid, benazolin, benazolin-ethyl, benfluralin, benfuresate, bensulfuron, bensulfuronmethyl, bensulide, bentazon, bentazonsodium, benzadox, benzipram, benzobicyclon, benzofenap, benzofluor, benzoylprop, benzthiazuron, bifenox, bilanafos, bilanafos-sodium, bispyribac-sodium, borax, bromacil, bromobonil, bromobutide, bromofenoxim, bromoxynil, bromoxynil heptanoate, bromoxynil octanoate, bromoxynil-potassium, brompyrazon, butachlor, butafenacil, butamifos, butenachlor, buthidazole, buthiuron, butralin, butroxydim, buturon, butylate,
cafenstrole, calcium cyanamide, cambendichlor, carbasulam, carbetamide, carboxazole, carfentrazone-ethyl, chlomethoxyfen, chloramben, chloranocryl, chlorazifop, chlorazine, chlorbromuron, chlorbufam, chloreturon, chlorfenac, chlorfenprop, chlorflurazole, chlorflurenol-methyl, chloridazon, chlorimuron, chlorimuron-ethyl, chlornitrofen, chloroacetic acid, chloropon, chlorotoluron, chloroxuron, chloroxynil, chlorprocarb, chlorpropham, chlorsulfuron, chlorthal, chlorthal-dimethyl, chlorthiamid, cinidon-ethyl, cinmethylin, cinosulfuron, clethodim, cliodinate, clodinafop, clodinafop-propargyl, clofop, clomazone, clomeprop, cloproxydim, clopyralid, clopyralid-olamine, cloransulam-methyl, CP 17029, credazine, cumyluron, cyanamide, cyanatryn, cyanazine, cycloate, cyclosulfamuron, cycloxydim, cycluron, cyhalofop-butyl, cyperquat, cyprazine, cyprazole, cypromid,
daimuron, dalapon, dalapon-sodium, dazomet, delachlor, desmedipham, desmetryn, di-allate, dicamba, dicamba-dimethylammonium, dicamba-potassium, dicamba-sodium, dichlobenil, dichloralurea, dichlormate, dichlorprop, dichlorprop-butotyl, dichlorprop-isoctyl, dichlorprop-dimethylammonium, dichlorprop-p, dichlorprop-potassium, diclofop, diclofop-methyl, diclosulam, diethamquat, diethatyl-ethyl, difenopenten, difenoxuron, difenzoquat, difenzoquat metilsulfate, diflufenican, diflufenzopyr, dimefuron, dimepiperate, dimethachlor, dimethametryn, dimethenamid, dimethipin, dimethylarsinic acid, dimexano, dimidazon, dinitramine, dinofenate, dinoprop, dinosam, dinoseb, dinoseb acetate, dinoterb, dinoterb acetate, dinoterb-diolamine, dinoterb-ammonium, diphenamid, dipropetryn, diquat, diquat dibromide, disul, dithiopyr, diuron, DMPA, DNOC, DSMA,
eglinazine-ethyl, ferrous sulfate, EL 177, endothal, epronaz, EPTC, erbon, esprocarb, ethalfluralin, ethametsulfuron-methyl, ethidimuron, ethiolate, ethofumesate, ethoxysulfuron, ethylene bis(trichloroacetate), etinofen, etnipromid, etobenzanid, EXD,
fenasulam, fenoprop, fenoxaprop-ethyl, fenoxaprop-P, fenoxaprop-P-ethyl, fenteracol, fenthiaprop, fentrazamide, fenuron, fenuron-TCA, flamprop-M, flamprop-M-isopropyl, flamprop-M-methyl, flamprop-methyl, flazasulfuron, florasulam, fluazifop, fluazifop-butyl, fluazifop-P, fluazifop-P-butyl, fluazolate, flucarbazone-sodium, fluchloralin, flufenacet, flufenican, flumetsulam, flumezin, flumiclorac, flumiclorac-pentyl, flumioxazin, flumipropyn, fluometuron, fluorodifen, fluoroglycofen, fluoroglycofen-ethyl, fluoromidine, fluoronitrofen, fluothiuron, flupoxam, flupropacil, flupropanate, flupropanate-sodium, flupyrsulfuron-methyl-sodium, flurenol, flurenol-butyl, fluridone, fluorochloridone, fluoroxypyr, fluoroxypyr-methyl, fluoroxypyr-2-butoxy-1-methylethyl, flurtamone, fluthiacet-methyl, FMC 19873, FMC 21844, FMC 21861, FMC 23486, FMC 25213, fomesafen, fomesafen-sodium, foramsulfuron, fosamine, fosamine-ammonium, furyloxyfen,
glufosinat, glufosinate-ammonium, glyphosate, glyphosate-trimesium, glyphosate-ammonium, glyphosate-isopropylammonium, glyphosate-sodium,
halosafen, halosulfuron, halosulfuron-methyl, haloxydine, haloxyfop, haloxyfop-etotyl, haloxyfop-P-methyl, HC-252, hexachloroacetone, hexafluoroacetone trihydrate, hexaflurate, hexazinone,
imazamethabenz, imazamethabenz-methyl, imazamox, imazapic, imazapyr, imazapyr-isopropylammonium, imazaquin, imazaquin-ammonium, imazethapyr, imazethapyr-ammonium, imazosulfuron, indanofan, iodobonil, iodosulfuron-methyl-sodium, ioxynil, ioxynil octanoate, ioxynil-sodium, ipazine, iprymidam, isocarbamid, isocil, isomethiozin, isonoruron, isopolinate, isopropalin, isoproturon, isouron, isoxaben, isoxaflutole, isoxapyrifop,
potassium cyanate, karbutilate,
lactofen, lenacil, linuron, LS830556,
MCPA, MCPA-butotyl, MCPA-2-ethylhexyl, MCPA-dimethylammonium, MCPA-potassium, MCPA-sodium, MCPA-thioethyl, MCPB, MCPB-ethyl, MCPB-sodium, mecoprop, mecoprop-P, medinoterb acetate, mefenacet, mefluidide, mesoprazine, mesosulfuron-methyl, mesotrione, metam, metam-sodium, metamifop, metamitron, metazachlor, metflurazon, methabenzthiazuron, methalpropalin, methazole, methiobencarb, methiuron, methometon, methoprotryne, methoxyphenone, methyl isothiocyanate, methylarsonic acid, methyldymron, metobenzuron, metobromuron, metolachlor, metosulam, metoxuron, metribuzin, metsulfuron, metsulfuron-methyl, MK-616, molinate, monalide, monisouron, monolinuron, monuron, morfamquat dichloride, MSMA,
naproanilide, napropamide, naptalam, naptalam-sodium, sodium chlorate, sodium chloroacetate, sodium dimethylarsinate, sodium pentachlorophenoxide, NC-330, neburon, nicosulfuron, nipyraclofen, nitralin, nitrofen, nitrofluorfen, nonanoic acid, norflurazon, noruron,
OCH, OCS 21693, oleic acid (fatty acids), orbencarb, oryzalin, oxadiargyl, oxadiazon, oxapyrazon, oxasulfuron, oxaziclomefone, oxyfluorfen,
parafluoron, paraquat, paraquat dichloride, pebulate, pendimethalin, pentachlorophenol, pentachlorophenyl laurate, pentanochlor, pentoxazone, perfluidone, pethoxamid, petroleum oils, phenisopham, phenmedipham, phenmedipham-ethyl, phenobenzuron, picloram, picolinafen, piperophos, polychlorodicyclopentadiene isomers, pretilachlor, primisulfuron, primisulfuron-methyl, procyazine, prodiamine, profluralin, profoxydim, proglinazine-ethyl, prometon, prometryn, propachlor, propanil, propaquizafop, propazine, propham, propisochlor, propoxycarbazone, propyzamide, prosulfalin, prosulfocarb, prosulfuron, proxan, prynachlor, pyraflufen-ethyl, pyrazolynate, pyrazosulfuron, pyrazosulfuron-ethyl, pyrazoxyfen, pyribenzoxim, pyributicarb, pyriclor, pyridate, pyriftalid, pyriminobac-methyl, pyrithiobac-sodium,
quinclorac, quinmerac, quinoclamine, quizalofop, quizalofop-ethyl, quizalofop-P, quizalofop-P-tefuryl, quizalofop-P-ethyl,
rhodethanil, rimsulfuron,
S-metolachlor, sebuthylazine, secbumeton, sethoxydim, siduron, simazine, simeton, simetryn, SMY 1500, sulcotrione, sulfallate, sulfentrazone, sulfometuron, sulfometuron-methyl, sulfosulfuron, sulfuric acid, sulglycapin, swep,
TCA-sodium, tebutam, tebuthiuron, tar oils, tepraloxydim, terbacil, terbucarb, terbuchlor, terbumeton, terbuthylazine, terbutryn, tetrafluoron, thenylchlor, thiazafluoron, thiazopyr, thidiazimin, thifensulfuron, thifensulfuron-methyl, thiobencarb, tiocarbazil, tioclorim, tralkoxydim, tri-allate, triasulfuron, triaziflam, tribenuron, tribenuron-methyl, tricamba, trichloroacetic acid, trichlorobenzyl chloride, triclopyr, triclopyr-butotyl, triclopyr-triethylammonium, tridiphane, trietazine, trifloxysulfuron, trifluralin, triflusulfuron, triflusulfuron-methyl, trifop, trifopsime, trimeturon, tripropindan, tritac,

### UBI-S734,

vernolate,
WL 9385 and/or xylachlor.

Examples of insecticides can be chosen from the group consisting of:

1,1-dichloro-1-nitroethane, 1,1-dichloro-2,2-bis(4-ethylphenyl)ethane, 1,2-dichloropropane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindane-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate,
abamectin, acephate, acetamiprid, acrinathrine, acrylonitrile, alanycarb, aldicarb, aldoxycarb, aldrin, allethrin [(1R)-isomers], allyxycarb, alpha-cypermethrin, aluminum phosphide, amidithion, amidothioate, aminocarb, amiton, amitraz, anabasine, athidathion, azadirachtin, azamethiphos, azinphos-ethyl, azinphos-methyl, azothoate,
bacillus thuringiensis delta-endotoxins, barium polysulfide, Bayer 22/190, Bayer 22408, bendiocarb, benfuracarb, bensultap, beta-cyfluthrin, beta-cypermethrin, bifenthrin, bioallethrin, bioallethrin s-cyclopentenyl isomer, biopermethrin, bioresmethrin, bis(2-chloroethyl)ether, bistrifluoron, borax, bromfenvinfos, bromocyclen, bromophos, bromophos-ethyl, bufencarb, buprofezin, butacarb, butathiofos, butocarboxim, butonate, butoxycarboxim,
cadusafos, calcium cyanide, calcium polysulfide, camphechlor, carbanolate, carbaryl, carbofuran, carbophenothion, carbosulfan, cartap, cartap hydrochloride, chlorbicyclen, chlordane, chlordecone, chlordimeform, chlorethoxyfos, chlorfenapyr, chlorfenvinphos, chlorfluazuron, chlormephos, chloropicrin, chlorphoxim, chlorprazophos, chlorpyrifos, chlorpyrifos-methyl, chlorthiophos, chromafenozide, cloethocarb, clothianidin, coumaphos, coumithoate, crotoxyphos, crufomate, cryolite, CS 708, cyanofenphos, cyanophos, cyanthoate, cycloprothrin, cyfluthrin, cyhalothrin, cypermethrin, cyphenothrin [(1R)-trans-isomers], cyromazine,
DAEP, dazomet, DDT, decarbofuran, deltamethrin, demephion, demeton-5-methyl, demeton-S-methylsulphone, demeton, diafenthiuron, dialifos, diamidafos, diazinon, dicapthon, dichlofenthion, dichlorvos, dicrotophos, dicyclanil, dieldrin, diethyl 5-methylpyrazol-3-yl phosphate, diflubenzuron, dimefox, dimethoate, dimethrin, dimethylvinphos, dimetilan, dinex, dinoseb, dinotefuran, diofenolan, dioxabenzofos, dioxacarb, dioxathion, disulfoton, dithicrofos, DNOC, DSP,
EI1642, emamectin, emamectin benzoate, EMPC, empenthrin [(EZ)-(1R)-isomers], endosulfan, endothion, endrin, ENT 92, EPBP, EPN, epofenonane, esfenvalerate, ethiofencarb, ethion, ethoate-methyl, ethoprophos, ethylene dibromide, ethylene dichloride, etofenprox, etrimfos,
famphur, fenchlorphos, fenethacarb, fenfluthrin, fenitrothion, fenobucarb, fenoxacrim, fenoxycarb, fenpirithrin, fenpropathrin, fensulfothion, fenthion, fenvalerate, fipronil, flonicamid, flucofuron, flucycloxuron, flucythrinate, fluenetil, flufenoxuron, flufenprox, flumethrin, fluvalinate, FMC 1137, fonofos, formetanate, formetanate hydrochloride, formothion, formparanate, fosmethilan, fospirate, fosthiazate, fosthietan, furathiocarb, furethrin,
gamma-HCH, GY-81,
halofenozide, HCN, heptachlor, heptenophos, hexaflumuron, hydramethylnon, hydroprene, hyquincarb,
imidacloprid, imiprothrin, indoxacarb, IPSP, isazofos, isobenzan, isodrin, isofenphos, isolan, isoprocarb, isopropyl O-(methoxyaminothiophosphoryl)salicylate, isothioate, isoxathion,
jodfenphos,
kelevan, kinoprene, carbon disulfide,
lambda-cyhalothrin, leptophos, lirimfos, lufenuron, lythidathion,
m-cumenyl methylcarbamate, magnesium phosphide, malathion, mazidox, mecarbam, mecarphon, menazon, mephosfolan, mesulfenfos, metam, metam-sodium, methacrifos, methamidophos, methanesulfonyl fluoride, methidathion, methiocarb, methocrotophos, methomyl, methoprene, methoquin-butyl, methothrin, methoxychlor, methoxyfenozide, methyl bromide, methyl isothiocyanate, metolcarb, metoxadiazone, mevinphos, mexacarbate, milbemectin, mipafox, mirex, monocrotophos, morphothion, naled, sodium cyanide, sodium fluoride, sodium hexafluorosilicate, sodium pentachlorophenoxide, sodium selenate, naphthalene, NC-170, nicotine, nifluridide, nitenpyram, nithiazine, nitrilacarb, nornicotine, novaluron,
O-2,5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidine-4-yl phosphorothioate, O,O,O′,O′-tetrapropyl dithiopyrophosphate, oleic acids (fatty acids), omethoate, oxamyl, oxydemeton-methyl, oxydeprofos, oxydisulfoton,
parathion, parathion-methyl, pentachlorophenol, pentachlorophenyl laurate, permethrin, petroleum oils, PH 60-38, phenkapton, phenothrin [(1R)-trans-isomer], phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, polychlorodicyclopentadiene isomers, polychloroterpene, prallethrin, primidophos, profenofos, promacyl, promecarb, propaphos, propetamphos, propoxur, prothiofos, prothoate, pymetrozine, pyraclofos, pyresmethrin, pyrethrins (chrysanthemates), pyrethrins (pyrethrates), pyrethrins (pyrethrum), pyridaben, pyridaphenthion, pyrimidifen, pyrimitate, pyriproxyfen,
mercury chloride, quinalphos, quinalphos-methyl, quinothion, quintiofos,
R-1492, resmethrin, rotenone, RU15525, RU 25475, ryanodine,
sabadilla, schradan, silafluofen, SN 72129, sophamide, spinosad, sulcofuron, sulcofuron-sodium, sulfluramid, sulfotep, sulfuryl fluoride, sulprofos,
tau-fluvalinate, tazimcarb, TDE, tebufenozide, tebupirimfos, tar oils, teflubenzuron, tefluthrin, temephos, TEPP, terallethrin, terbufos, tetrachloromethane, tetrachlorvinphos, tetramethrin, tetramethrin [(1R)-isomers], theta-cypermethrin, thiacloprid, thiamethoxam, thicrofos, thiocarboxime, thiocyclam, thiodicarb, thiofanox, thiometon, thionazin, thiosultap-sodium, tolfenpyrad, tralomethrin, transfluthrin, transpermethrin, triamiphos, triazamate, triazophos, trichlorfon, trichloronat, trifenofos, triflumuron, trimethacarb, triprene,
vamidothion,
XMC, xylylcarb,
zeta-cypermethrin, zinc phosphide, zolaprofos and/or ZXI 8901.

Examples of fungicides can be chosen from the group consisting of

(R,S)—N-(3,5-dichlorophenyl)-2-(methoxymethyl)succinimide, 1,3-dichloro-1,1,3,3-tetrafluoro-acetone hydrate, 1-chloro-2,4-dinitronaphthalene, 1-chloro-2-nitropropane, 1-hydroxy-1H-pyridine-2-thione, 2,3-dihydro-5-phenyl-1,4-dithiin 1,1,4,4-tetraoxide, 2-(2-heptadecyl-2-imidazolin-1-yl)ethanol, 2-methoxyethylmercury acetate, 2-methoxyethylmercury chloride, 2-methoxyethylmercury silicate, 2-phenylphenol, 3-(4-chlorophenyl)-5-methylrhodanine, 4-(2-nitroprop-1-enyl)phenyl thiocyanate, 5-methyl-6-thioxo-1,3,5-thiadiazinan-3-ylacetic acid, 8-hydroxyquinoline sulfate,
ampropylfos, anilazine, azaconazole, azithiram, azoxystrobin,
barium polysulfide, Bayer 32394, benalaxyl, benodanil, benomyl, benquinox, bentaluron, benzamacril, benzamorf, binapacryl, biphenyl, bis(methylmercury) sulfate, bis(tributyltin) oxide, bitertanol, blasticidin-s, borax, Bordeaux mixture, bromuconazole, bupirimate, buthiobate,
cadmium calcium copper tin chromate sulfate, calcium polysulfide, captafol, captan, carbamorph, carbendazim, carboxin, carpropamid, CECA, CGA 80 000, chinomethionat, chlobenthiazone, chloraniformethan, chloranil, chlorfenazole, chloroneb, chlorothalonil, chlorquinox, chlozolinate, climbazole, cufraneb, cuprobam, cyazofamid, cyclafuramid, cycloheximide, cymoxanil, cypendazole, cyproconazole, cyprodinil, cyprofuram,
dazomet, debacarb, decafentin, dehydroacetic acid, dichlofluanid, dichlone, dichlorophen, dichlozoline, diclobutrazol, diclocymet, diclomezine, dicloran, diethofencarb, difenoconazole, difenzoquat, difenzoquat metilsulfate, diflumetorim, dimethirimol, dimethomorph, diniconazole, diniconazole-M, dinobuton, dinocap, dinocton, dinopenton, dinosulfon, dinoterbon, diphenylamine, dipyrithione, ditalimfos, dithianon, dodemorph, dodemorph acetate, dodicin, dodine, dodine free base, drazoxolon,
EBP, edifenphos, epoxiconazole, ESBP, etaconazole, etem, ethaboxam, ethirimol, ethoxyquin, etridiazole,
famoxadone, fenamidone, fenaminosulf, fenapanil, fenarimol, fenbuconazole, fenfuram, fenhexamid, fenitropan, fenoxanil, fenpiclonil, fenpropidin, fenpropimorph, fentin, fentin acetate, fentin hydroxide, ferbam, ferimzone, fluazinam, fludioxonil, fluoroimide, fluotrimazole, fluquinconazole, flusilazole, flusulfamide, flutolanil, flutriafol, folpet, formaldehyde, fosetyl, fosetyl-aluminum, fuberidazole, furalaxyl, furametpyr, furcarbanil, furconazole, furconazole-cis, furmecyclox, furophanate,
gliotoxin, glyodin, griseofulvin, guazatine, guazatine acetate, GY-81,
halacrinate, Hercules 3944, hexachlorobenzene, hexaconazole, hexylthiofos, hymexazole,
ICIA0858, imazalil, imazalil sulfate, imibenconazole, iminoctadine, iminoctadine triacetate, iminoctadine tris(albesilate), ipconazole, iprobenfos, iprodione, iprovalicarb, isopamphos, isoprothiolane, isovaledione,
kasugamycin, kasugamycin hydrochloride hydrate, kresoxim-methyl, copper bis(3-phenylsalicylate), copper hydroxide, copper hydrazinium sulfate, copper octanoate, copper oxide, copper oxychloride, copper sulfate, copper sulfate (tribasic), copper zinc chromate,
mancopper, mancozeb, maneb, mebenil, mecarbinzid, mepanipyrim, mepronil, metalaxyl, metalaxyl-m, metam, metam-sodium, metazoxolon, metconazole, methasulfocarb, methfuroxam, methyl isothiocyanate, methylarsenic sulfide, methylmercury dicyandiamide, metiram, metominostrobin, metsulfovax, mildiomycin, milneb, mucochloric anhydride, myclobutanil, myclozolin,
N-3,5-dichlorophenylsuccinimide, N-3-nitrophenylitaconimide, N-ethylmercury-4-toluenesulfonanilide, nabam, naphthalene, natamycin, nickel bis(dimethyldithiocarbamate), nitrothal-isopropyl, nuarimol,
OCH, octhilinone, oleic acid (fatty acids), ofurace, oxadixyl, oxine-copper, oxpoconazole fumarate, oxycarboxin,
pefurazoate, penconazole, pencycuron, pentachlorophenol, pentachlorophenyl laurate, phenylmercury acetate, phenylmercury dimethyldithiocarbamate, phenylmercury nitrate, phosdiphen, phosphonic acid (phosphoric acid), phthalide, picoxystrobin, piperalin, polyoxin B, polyoxins, polyoxorim, potassium hydroxyquinoline sulfate, probenazole, prochloraz, procymidone, propamocarb, propamocarb hydrochloride, propiconazole, propineb, prothiocarb, pyracarbolid, pyraclostrobin, pyrazophos, pyributicarb, pyridinitril, pyrifenox, pyrimethanil, pyroquilon, pyroxychlor, pyroxyfur,
quinacetol, quinazamid, quinconazole, quinoxyfen, quintozene, mercury(I) chloride, mercury(II) chloride, mercury oxide,
rabenzazole,
salicylanilide, sulfur, sec-butylamine, silthiofam, simeconazole, sodium 2-phenylphenoxide, sodium pentachlorophenoxide, spiroxamine, SSF-109, sultropen, SYP-L190,
tebuconazole, tecnazene, tecoram, tar oils, tetraconazole, thiabendazole, thiadifluor, thicyofen, thifluzamide, thiochlorfenphim, thiophanate, thiophanate-methyl, thioquinox, thiram, tioxymid, tolclofos-methyl, tolylfluanid, triadimefon, triadimenol, triamiphos, triarimol, triazbutil, triazoxide, trichlamide, tricyclazole, tridemorph, trifloxystrobin, triflumizole, triforine, triticonazole,
urbacid,
validamycin, vinclozolin,

### XRD-563,

zarilamid, zineb, ziram and/or zoxamide.

Examples of nematicides can be chosen from the group consisting of:

1,2-dibromo-3-chloropropane, 1,2-dichloropropane with 1,3-dichloropropene, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid,
aldicarb, aldoxycarb,
bacillus firmus,
cadusafos, carbofuran,
chloropicrin, cloethocarb,
dazomet, DCIP, diamidafos, dichlofenthion,
ethoprophos, ethylene dibromide,
fenamiphos, fensulfothion, fosthiazate, fosthietane,

### GY-81,

isamidofos, isazofos,
kinetin, carbon disulfide,
metam, metam-sodium, methyl isothiocyanate, myrothecium verrucaria composition,
oxamyl,
phorate,
terbufos, tetrachlorothiophene, thionazine and/or triazophos.

Examples of molluscicides can be chosen from the group consisting of:

iron phosphate,
fentin, fentin acetate, fentin hydroxide,
metaldehyde, methiocarb,
niclosamide, niclosamide-olamine,
tazimcarb, thiodicarb, trifenmorph and/or trimethacarb.

Examples of growth regulators can be chosen from the group consisting of:

1-naphthylacetic acid, 2,3-dihydro-5,6-diphenyl-1,4-oxathiin, 2-(1-naphthyl)acetamide, 2-cyano-3-(2,4-dichlorophenyl)acrylic acid, 2-hydrazinoethanol, 2-naphthyloxyacetic acid, 4-CPA, 4-indol-3-ylbutyric acid, 6-benzylaminopurine,
AC 94,377, alorac, amidochlor, aminoethoxyvinylglycine, ancymidol,
BTS 44584, buminafos, butralin,
calcium cyanamide, carbaryl, carvone, chlorfluren, chlorflurenol-methyl, chlormequat, chlormequat chloride, chlorphonium chloride, chlorpropham, ciobutide, clofencet, clofencet potassium, cloprop, cloxyfonac, cloxyfonac-sodium, cyanamide, cyclanilide, cycloheximide, cytokinins,
daminoazide, dicamba-methyl, dichlorflurenol, dikegulac, dikegulac-sodium, dimethipin, dimexano,
endothal, etacelasil, ethephon, ethychlozate, ethyl 1-naphthylacetate,
fenoprop, fenridazon, flumetralin, fluoridamid, flurprimidol, forchlorfenuron,
gibberellic acid, gibberellin A4, gibberellin A4 with gibberellin A7, gibberellin A7, glyphosine,
heptopargil, hexafluoroacetone trihydrate, holosulf,
inabenfide, indol-3-ylacetic acid, isoprothiolane, isopyrimol,
kinetin,
maleic hydrazide, maleic hydrazide potassium salt, MCPB-ethyl, mefluidide, mepiquat, mepiquat chloride, methasulfocarb,
N-(2-ethyl-2H-pyrazol-3-yl)-N′-phenylurea, N-m-tolylphthalamic acid, N-pyrrolidinylsuccinamic acid, N-decanol, N-phenylphthalamic acid, nitrophenolate mixture, nonanoic acid,
paclobutrazol, piproctanyl bromide, prohexadione, prohexadione-calcium, propham, propyl 3-tert-butylphenoxyacetate, pydanon,
sintofen, sodium (Z)-3-chloroacrylate,
tecnazene, tetcyclacis, thidiazuron, triapenthenol, tribufos, tributyl phosphorotrithioate, trinexapac, trinexapac-ethyl,
uniconazole and/or uniconazole-P.

Examples of bird repellants can be chosen from the group consisting of 4-aminopyridine, anthraquinone, methiocarb and/or ziram.

In an additional embodiment of the present invention the active substances are agents for the healing, alleviation or prevention of diseases of man or animals, preferably chosen from the group consisting of acidosis therapeutic agents, analeptics/antihypoxemics, analgesics/antirheumatics, anthelmintics, antiallergics, antianemics, antiarrhythmics, antibiotics/antiinfectives, antidementia, antidiabetics, antidotes, antiemetics/antivertigo, antiepileptics, antihemorrhagics, antihypertensives, antihypoglycemics, antihypotensives, anticoagulants, antimycotics, antiparasitic agents, antiinflammatories, antitussives/expectorants, arteriosclerosis drugs, bronchodilators/antiasthmatics, cholagogues and biliary therapeutic agents, cholinergics, corticoids, dermatologicals, diuretics, blood flow-promoting agents, anticraving agents/agents for the treatment of addictive disorders, enzyme inhibitors, preparations for enzyme deficiency and transport proteins, fibrinolytics, geriatric agents, antigouts, gynecologicals, hepatics, hypnotics/sedatives, immunomodulators, cardiac drugs, coronary agents, laxatives, lipid-lowering agents, local anesthetics/neurotherapeutic agents, gastrointestinal drugs, antimigraines, muscle relaxants, opthalmologicals, osteoporosis drugs/calcium metabolism regulators, otologicals, psychoactive drugs, rhinologicals/sinusitis drugs, roborants/tonics, thyroid therapeutic agents, sex hormones and their inhibitors, spasmolytics/anticholinergics, platelet aggregation inhibitors, antituberculars, stimulants, urologicals, vein therapeutic agents, cytostatics, other antineoplastics and protectives, mineral substances, food supplements and/or vitamins, particularly preferably boldine, quinolones, felodipine, flurbiprofen, ibuprofen, ketoprofen, macrolides, nicardipine, nifedipine, nimodipine, nisoldipine, nitrendipine, norfloxacin, ofloxacin, paclitaxel, sulfonamides and/or tetracyclines. The abovementioned substance categories cover the active substances most frequently used in human medicine, veterinary medicine and over-the-counter (OTC) compositions and are accordingly the most asked after by the user. It is possible to have present, in the individual compartments of the molded article according to the invention, in each case active substances of the same substance category but also to use, in the individual compartments, active substances from different categories.

In an additional embodiment of the present invention, the filler is chosen from the group consisting of minerals, preferably clay minerals and/or colloidal silica, particularly preferably kaolin, kaolinite, halloysite, montmorillonite, talc, bentonite, vermiculite and/or allophane. The term “minerals” generally comprises oxides, hydroxides, silicates, carbonates and sulfates of calcium, magnesium, aluminum and titanium. Such minerals are chemically inert for the intended purpose, cheap and available in large amounts. Due to their ability to incorporate water, satisfactorily processable pastes can be used for the preparation of the molded article according to the invention. In addition, these minerals are sufficiently nonabrasive enough not to attack processing tools and machinery.

In an additional embodiment of the present invention, the filler exhibits a particle size having a d₉₀ value for the volume-weighted distribution of ≧0.1 μm to ≦1000 μm, preferably ≧0.5 μm to ≦500 μm, more preferably ≧1 μm to ≦50 μm. Particles with such sizes can, on the other hand, be satisfactorily processed in the preparation of the molded article according to the invention, since they are flowable or pumpable in the dry state and in the form of a paste with plasticizer. On the other hand, particles of such a size are advantageous for the user, since the does not have to worry about damage caused by relatively coarse particles to a spray device or other device for the further processing. The d₉₀ value of the volume-weighted distribution is that particle size for which 90% of the particle volume is formed of particles smaller than or the same as the d₉₀ value. Measurement methods for the determination of the volume-weighted distribution are listed, for example, in Terence Allen Particle Size Measurement, Kluwer Academic Publishers, Dordrecht/Boston/London, 1999. The depiction of the results is likewise mentioned in this literature reference or can also take place according to the standard DIN ISO 9276-1.

In an additional embodiment of the present invention, the matrix furthermore comprises auxiliaries, chosen from the group consisting of binders, synthetic macromolecules, anionic surfactants, nonionic surfactants, lipids with an ester linkage, bursting agents, organic liquids and/or water.

Binders have, inter alia, the role of holding together individual constituents of the molded article in a dimensionally stable and processable fashion. Examples of binders can be chosen from the group consisting of cellulose derivatives, microcrystalline cellulose, sodium carboxymethylcellulose, methylcellulose, hydroxypropylcellulose, hydroxypropylmethylcellulose, lactose, starch (wheat, maize, potato, rice starch), starch derivatives, sucrose, glucose, mannitol, sorbitol, dicalcium phosphate, tricalcium phosphate, bole, zinc oxide, gelatin, maltodextrins, polysaccharides, oligosaccharides, stearic acid, calcium stearate, shellac, cellulose acetate phthalate and/or hydroxymethylcellulose phthalate.

Examples of synthetic macromolecules can be chosen from the group consisting of copolymers of dimethylaminomethacrylic acid and neutral methacrylic acid esters, acrylic and methacrylic acid ester copolymers with trimethylammoniomethyl acrylate, polymers of methacrylic acid and methacrylic acid esters, acrylic acid ethyl ester/methacrylic acid methyl ester copolymer, methacrylic acid/acrylic acid methyl ester copolymer, polyvinylpyrrolidone, polyvinylpyrrolidone vinyl acetate and/or polyvinyl alcohols.

Examples of anionic surfactants can be chosen from the group consisting of soaps, salts of fatty acids, sodium palmitate, sodium stearate, sodium oleate, sodium salts of fatty alcohol sulfates, sulfosuccinates, alkylnaphthalenesulfonates and/or alkyl sulfates.

Examples of nonionic surfactants can be chosen from the group consisting of partial fatty acid esters of polyvalent alcohols, partial fatty acid esters of sorbitans, partial fatty acid esters of polyhydroxyethylene sorbitan, polyhydroxyethylene fatty alcohol ethers, polyhydroxyethylene fatty acid esters, ethylene oxide/propylene oxide block copolymers, ethoxylated triglycerides and/or silicone surfactants.

Examples of lipids with an ester linkage can be chosen from the group consisting of glycerides, oils, hydrogenated oils, semisynthetic and synthetic glycerides, solid and semisolid waxes, liquid waxes and/or phosphatides.

Examples of disintegrating agents can be chosen from the group consisting of water-soluble salts, crosslinked polyvinylpyrrolidone and/or mixtures of sugars, sodium hydrogencarbonate and citric acid.

Examples of organic liquids can be chosen from the group consisting of silicone oils, glycerol, ethanol, isopropanol, propylene glycol and/or polypropylene glycols.

These auxiliaries support the rapid disintegration of the molded article when charged to water and the dispersing of water-insoluble active substances in a spray slurry, an ointment or a drink. The choice of these disintegrating agents renders superfluous bursting agents which allow the molded article to disintegrate with evolution of gas. Formation of foam in the container provided for the application of the spray slurry, the ointment or the drink is accordingly avoided.

In an additional embodiment of the present invention the molded article comprises an inner compartment and an outer compartment, the outer compartment at least partially enveloping the inner compartment. It is possible, through this, to introduce for example light-sensitive substances into the inner compartment and to shield these by means of the outer compartment. Protection of the user can likewise thus be increased if the active substance more toxic to the user is moved to the inside and thus contact cannot occur.

In an additional embodiment of the present invention the molded article is a coextrudate of at least two different compartments. Coextrudates can be prepared on commercially available devices and can, through the choice of the extrusion conditions, be obtained as granules with precisely defined dimensions. Uniform dimensions render the granules easier to flow.

In an additional embodiment of the present invention, the liquid content of the ready-for-sale molded article is ≧0.1% by weight to ≦50% by weight, preferably ≧1% by weight to ≦40% by weight, particularly preferably ≧5% by weight to ≦25% by weight. In the context of the present invention the term “ready-for-sale” means in this connection that all preparation and treatment steps of the molded article are complete. Molded articles according to the invention with these liquid contents prove their worth in handling. They are neither too dry, which can result in breaking and development of dust during transportation, storage or metering. Nor are they too wet, which might cause the individual molded articles to adhere to one another and therefore to be less flowable. Liquids can in this connection be both organic liquids, such as, for example, ethanol or isopropanol, or water may be involved in this connection. If the molded article comprises several liquids, the liquid content mentioned is the sum of the contents of the individual liquids.

A subject matter of the present invention is likewise a process for the preparation of the abovedescribed molded article, comprising the steps:
a) mixing a first active substance composition and a first matrix in a first mixer
b) mixing a second active substance composition and a second matrix in the first or in a second mixer
c) adding liquid to the mixture of a first active substance composition and of a first matrix
d) adding liquid to the mixture of a second active substance composition and of a second matrix
e) spatially separated extrusion of the mixture from steps a) and c), on the one hand, and of the mixture from steps b) and d), on the other hand
f) bringing together the individual extrudates from step e) to give an extrudate/molded article
g) dividing the extrudate/molded article obtained into portions
h) drying the extrudate/molded article obtained,
   the matrices exhibiting, after drying, a filler content of ≧20% by weight to ≦100% by weight.

The first active substance composition in step a) can be a mixture of different active substances or an individual active substance. After step a) this can exhibit, with regard to the first matrix, a ratio by weight of ≧0.01% by weight to ≦99.99% by weight, preferably ≧1% by weight to ≦50% by weight, particularly preferably ≧2% by weight to ≦20% by weight. It is also possible for the mixer to be part of the extruder or for the extruder to undertake all or part of the duty of the mixing.

The second active substance composition in step b) can be a mixture of different active substances an individual active substance. After step b) this can exhibit, with regard to the second matrix, a ratio by weight of ≧0.01% by weight to ≦99.99% by weight, preferably ≧1% by weight to ≦50% by weight, particularly preferably ≧2% by weight to ≦20% by weight. It is also possible for the mixer to be part of the extruder or for the extruder to undertake all or part of the duty of the mixing.

The liquid added in step c) can be both an organic liquid, such as, for example, silicone oils, glycerol, ethanol, isopropanol, propylene glycol or polypropylene glycols; it can in this connection concern water or liquid mixtures. It can exhibit a proportion of the total weight of the composition of ≧5% by weight to ≦60% by weight, preferably ≧10% by weight to ≦50% by weight, particularly preferably ≧15% by weight to ≦40% by weight.

The liquid added in step d) can be both an organic liquid, such as, for example, silicone oils, glycerol, ethanol, isopropanol, propylene glycol or polypropylene glycols; it can in this connection concern water or liquid mixtures. It can exhibit a proportion of the total weight of the composition of ≧5% by weight to ≦60% by weight, preferably ≧10% by weight to ≦50% by weight, particularly preferably ≧15% by weight to ≦40% by weight.

Lower liquid contents in steps c) and d) are detrimental to the plasticity of the composition, so that the extrusion step is disadvantageously affected. Higher liquid contents reduce the stability of the extruded molded article and result in an unnecessarily high energy use in the final drying.

The spatially separated extrusion of the mixture from steps a) and c), on the one hand, and of the mixture from steps b) and d), on the other hand, can be carried out in conventional extruders exhibiting several die orifices. Single-screw extruders may be involved in this connection. However, multi-screw extruders, for example, twin-screw extruders, may also be involved. The multi-screw extruders may exhibit corotating or counterrotating screws. These screws may be arranged in parallel or tapered fashion with respect to one another. The screws may be composed of conveying components, mixing and/or kneading components and components for increasing the pressure. The second mixture can advantageously be transported using a positive displacement pump or by the action of the extruder.

The molded article is brought to completion by bringing together the individual extrudates from step e) to give an extrudate/molded article, as described in step f).

In the next step g), the extrudate/molded article obtained is divided into portions. For example, either open coextrudates can be obtained, by means of a guillotine or rotating blades, or closed coextrudates can be obtained, by means of a pillow cutter. The pillow cutter pinches the individual extrudate sections to give pillows, the covering on the pinching points being placed over the core. This results in closed coextrudates. It is likewise possible for the individual portions after dividing into portions, for example with a pillow cutter, to be still connected together. This is advantageous if the relatively large units are to be dried and only subsequently through breaking are to be subdivided into individual pillows. The length of the molded article portion from the dividing is advantageously ≧0.5 mm to ≦80 mm, preferably ≧1 mm to ≦20 mm, particularly preferably ≧2 mm to ≦7 mm. Granules with such dimensions exhibit an advantageous ratio of surface area to volume, so that the decomposition and the redispersing in water can proceed rapidly.

As concluding step, the extrudate/molded article obtained is dried. The liquid content can in this connection be brought to ≧0.1% by weight to ≦50% by weight, preferably ≧1% by weight to ≦40% by weight, particularly preferably ≧5% by weight to ≦25% by weight. The advantages of a liquid content in this range have already been described above.

The process according to the invention is distinguished in that the matrices after the drying exhibit a filler content of ≧20% by weight to ≦100% by weight. The advantages of this procedure have already been described above.

It is, though, likewise possible for the matrices after the drying to exhibit a filler content of ≧40% by weight to ≦90% by weight or ≧70% by weight to ≦80% by weight.

In an embodiment of the present process according to the invention, the extruding in step e) is carried out at a pressure of ≧1 bar to ≦300 bar, preferably ≧2 bar to ≦150 bar, particularly preferably ≧8 bar to ≦80 bar. The pressure is, in this connection, understood as absolute pressure, so that a pressure of 1 bar is to be regarded as without the use of pressure. The indication of the pressure refers to the pressure in the extruder on the die, and thus to the discharge pressure. It is accordingly possible, with high solid contents of the material to be extruded, to achieve a simultaneously high processing rate.

In an additional embodiment of the present invention, a hollow strand, on the one hand, and a core extrudate, on the other hand, are formed in step e) and these are brought together in step f) by introducing the core into the hollow strand. Core/shell extrudates can be produced by this procedure. The coextrusion and the introduction are advantageously carried out in one appliance. It is advisable for the coextrusion and the bringing together to be carried out in a matrix or a die head. In principle, all conceivable extruder dies which produce a hollow strand can be used. The die can be shaped with rotational symmetry or with another shape. The profile of the hollow strand can, for example, exhibit an external diameter, measured at the thickest point, of ≧1 mm to ≦20 mm, preferably ≧3 mm to ≦10 mm, particularly preferably ≧5 mm to ≦7 mm. The wall thickness of the hollow strand, measured at the thickest point, can lie in a range from ≧0.5 mm to ≦19 mm, preferably ≧0.7 mm to ≦10 mm, particularly preferably ≧1 mm to ≦2 mm.

The present invention furthermore relates to the use of molded articles according to the invention in the preparation of combination products for agrochemicals or agents for the healing, alleviation or prevention of diseases of man or animals. By way of example, granules can be prepared with which the end user can safely and simply prepare spray slurries for the treatment of agricultural effective areas.

The following examples 1 and 2 should give a fuller explanation of the present invention.

Use is made, for the assessment of the redispersibility of granules, of a test apparatus consisting of a cylindrical feed vessel (height: 70 mm; diameter: 135 mm) in which is a centrally positioned sieving unit (height: 33 mm, diameter: 100 mm, mesh width: 200 μm) standing on four supports (height: 2 mm). For the dispersing test, 600 ml of distilled water (temperature: 20° C.) are introduced into the feed vessel and 6 g of granules are introduced into the sieving unit. A circulating flow is produced using a three-bladed propeller stirrer (diameter: 55 mm; rotational speed: 250 rev/min) and a stirring drive of the IKA Werk RW-20 type, the flow in the sieving unit being directed downward and the flow in the annular gap between sieving unit and feed vessel being directed upward. The stirrer is positioned approximately 10 mm above the sieve netting. In order to analyze the dispersing time of the granules, the electrical conductivity of the solution is continuously measured and plotted against the time. After at the latest 10 minutes, the dispersing experiment is halted and the sieve residue is subsequently evaluated by inspection.

### EXAMPLE 1

Formulation 1: the following components were charged for a core/shell coextrusion:

| Ingredient | Proportion [% by weight] |
|---|---|
| Shell: | |
| Triabon | 0.7 |
| Kaolin | 47.0 |
| Lignosulfonate | 17.6 |
| PVP (polyvinylpyrrolidone) | 3.5 |
| Sodium diisopropylnaphthalenesulfonate | 1.4 |
| Water | 29.8 |
| Core: | |
| Fenoxaprop-P-ethyl | 1.0 |
| Kaolin | 55.0 |
| Lignosulfonate | 15.0 |
| Sodium diisopropylnaphthalenesulfonate | 2.0 |
| Glycerol | 8.1 |
| Water | 18.9 |

The following processing parameters were used for the extrusion:

| | |
|---|---|
| Shell: | |
| Die pressure: | 53 bar |
| Temperature: | 37° C. |
| Screw speed: | 300 revolutions/minute |
| Core: | |
| Die pressure: | 66 bar |

The molded articles obtained were dried in a drying cupboard at 50° C. at standard pressure for 4 hours.

The redispersibility according to the test described above was carried out on molded articles with a length of 2 mm, a diameter of the shell of 4 mm and a diameter of the core of 2 mm. After less than 600 seconds, a plateau value for the conductivity was achieved. No residue could be determined on the sieve by optical monitoring.

### EXAMPLE 2

Formulation 2: the following components were charged for a core/shell coextrusion:

| Ingredient | Proportion [% by weight] |
|---|---|
| Shell: | |
| Thiacloprid | 0.7 |
| Kaolin | 47.0 |
| Lignosulfonate | 17.6 |
| PVP (polyvinylpyrrolidone) | 3.5 |
| Sodium diisopropylnaphthalenesulfonate | 1.4 |
| Water | 29.8 |
| Core: | |
| Imidacloprid | 1.0 |
| Kaolin | 56.0 |
| Lignosulfonate | 15.0 |
| Sodium diisopropylnaphthalenesulfonate | 2.0 |
| Glycerol | 7.8 |
| Water | 18.2 |

The following processing parameters were used for the extrusion:

| | |
|---|---|
| Shell: | |
| Die pressure: | 39 bar |
| Temperature: | 32° C. |
| Screw speed: | 300 revolutions/minute |
| Core: | |
| Die pressure: | 36 bar |

The molded articles obtained were dried in a drying cupboard at 30° C. at standard pressure for 8 hours.

The redispersibility according to the test described above was carried out on molded articles with a length of 2 mm, a diameter of the shell of 4 mm and a diameter of the core of 2 mm. After less than 300 seconds, a plateau value for the conductivity was achieved. No residue could be determined on the sieve by optical monitoring.

1. A molded article comprising active substances, which comprises at least two compartments with different material composition, each compartment exhibiting, independently of one another, at least one active substance, wherein the active substance is present in the individual compartments in each case in a matrix and the respective matrix comprises at least one filler, the filler content of the respective matrix, based on the total weight of the respective matrix, coming to ≧20% by weight to ≦100% by weight.

2. The molded article as claimed in claim 1, the total weight of the matrices, based on the total weight of the molded article, coming to ≧0.01% by weight to ≦99.99% by weight.

3. The molded article as claimed in claim 1, the molded article comprising at least one first and one second compartment and the ratio by weight of the two compartments to one another coming to a ratio of ≧1:1 to ≦99:1.

4. The molded article as claimed in claim 1, the molded article comprising at least one first and one second compartment, the ratio by weight of the total amount of the active substances to the total amount of the fillers in the first compartment coming to ≧0.01:99.99 to ≦99.99:0.01, and the ratio by weight of the total amount of the active substances to the total amount of the fillers in the second compartment coming to ≧0.01:99.99 to ≦99.99:0.01.

5. The molded article as claimed in claim 1, the active substances being agrochemical active substances selected from the group consisting of herbicides, insecticides, fungicides, nematicides, pesticides, molluscicides, acaricides, fertilizers, growth regulators, safeners, bioenhancers and bird repellants.

6. The molded article as claimed in claim 1, the active substances being agents for the healing, alleviation or prevention of diseases of man or animals selected from the group consisting of acidosis therapeutic agents, analeptics/antihypoxemics, analgesics/antirheumatics, anthelmintics, antiallergics, antianemics, antiarrhythmics, antibiotics/antiinfectives, antidementia, antidiabetics, antidotes, antiemetics/antivertigo, antiepileptics, antihemorrhagics, antihypertensives, antihypoglycemics, antihypotensives, anticoagulants, antimycotics, antiparasitic agents, antiinflammatories, antitussives/expectorants, arteriosclerosis drugs, bronchodilators/antiasthmatics, cholagogues and biliary therapeutic agents, cholinergics, corticoids, dermatologicals, diuretics, blood flow-promoting agents, anticraving agents/agents for the treatment of addictive disorders, enzyme inhibitors, preparations for enzyme deficiency and transport proteins, fibrinolytics, geriatric agents, antigouts, gynecologicals, hepatics, hypnotics/sedatives, immunomodulators, cardiac drugs, coronary agents, laxatives, lipid-lowering agents, local anesthetics/neurotherapeutic agents, gastrointestinal drugs, antimigraines, muscle relaxants, opthalmologicals, osteoporosis drugs/calcium metabolism regulators, otologicals, psychoactive drugs, rhinologicals/sinusitis drugs, roborants/tonics, thyroid therapeutic agents, sex hormones and their inhibitors, spasmolytics/anticholinergics, platelet aggregation inhibitors, antituberculars, stimulants, urologicals, vein therapeutic agents, cytostatics, other antineoplastics and protectives, mineral substances, food supplements and vitamins.

7. The molded article as claimed in claim 1, the filler being selected from the group consisting of minerals.

8. The molded article as claimed in claim 1, the filler exhibiting a particle size having a d₉₀ value for the volume-weighted distribution of ≧0.1 μm to ≦1000 μm.

9. The molded article as claimed in claim 1, the matrix furthermore comprising auxiliaries selected from the group consisting of binders, synthetic macromolecules, anionic surfactants, nonionic surfactants, lipids with an ester linkage, bursting agents, organic liquids and water.

10. The molded article as claimed in claim 1, comprising an inner compartment and an outer compartment, the outer compartment at least partially enveloping the inner compartment.

11. The molded article as claimed in claim 1, the molded article being a coextrudate of at least two different compartments.

12. The molded article as claimed in claim 1, the liquid content of the ready-for-sale molded article being from ≧0.1% by weight to ≦50% by weight.

13. A process for the preparation of the molded article as claimed in claim 1, comprising the steps:
a) mixing a first active substance composition and a first matrix in a first mixer;
b) mixing a second active substance composition and a second matrix in the first or in a second mixer;
c) adding liquid to the mixture of a first active substance composition and of a first matrix;
d) adding liquid to the mixture of a second active substance composition and of a second matrix;
e) spatially separated extrusion of the mixture from steps a) and c), on the one hand, and of the mixture from steps b) and d), on the other hand;
f) bringing together the individual extrudates from step e) to give an extrudate/molded article;
g) dividing the extrudate/molded article obtained into portions; and
h) drying the extrudate/molded article obtained;
the matrices exhibiting, after drying, a filler content of ≧20% by weight to ≦100% by weight.

14. The process as claimed in claim 13, the extruding in step e) being carried out at a pressure of ≧1 bar to ≦300 bar.

15. The process as claimed in claim 13, a hollow strand, on the one hand, and a core extrudate, on the other hand, being formed in step e) and these two being brought together in step f) by introducing the core into the hollow strand.

16. A method of using the molded article as claimed in claim 1 in the preparation of combination products for agrochemicals or agents for the healing, alleviation or prevention of diseases of man or animals.
